(19) United States
(12) Patent Application Publication
(10) Pub. No.: US 2022/0415509 A1
(43) Pub. Date: Dec. 29, 2022

(54) SYSTEMS AND METHODS FOR MODELLING A HUMAN SUBJECT

(71) Applicant: KONINKLIJKE PHILIPS N.V., Eindhoven (NL)

(72) Inventors: Murtaza Bulut, Eindhoven (NL); Lieke Gertruda Elisabeth Cox, Eindhoven (NL); Cornelis Petrus Hendriks, Eindhoven (NL); Valentina Lavezzo, Heeze (NL)

(21) Appl. No.: 17/848,552

(22) Filed: Jun. 24, 2022

Related U.S. Application Data
(60) Provisional application No. 63/214,492, filed on Jun. 24, 2021.

(51) Int. Cl. G16H 50/20; G06N 3/08

(52) U.S. Cl. CPC G16H 50/20; G06N 3/08

(57) ABSTRACT

Systems and methods are proposed for controlling a Digital Twin of a biological asset of a subject based on: (i) characteristics of a user of the DT; (ii) a status of the subject; and (iii) previous usage of the DT. Such control is facilitated through the generation of one or more parameter values for the DT. For instance, the parameter value(s) may define one or more elements or components of the DT, such as program code, input requirements, output characteristics or a value of a tunable element of the DT.

### CROSS-REFERENCE TO RELATED APPLICATIONS

This patent application claims the priority benefit under 35 U. S. C. § 119(e) of U.S. Provisional Application No. 63/214,492, filed on Jun. 24, 2021, the contents of which are herein incorporated by reference.

### FIELD OF THE INVENTION

The invention relates to modelling human subjects, and more particularly to the field of models of biological function (commonly referred to as digital twins).

### BACKGROUND OF THE INVENTION

A recent development in healthcare is the so-called ‘digital twin’ concept. In this concept, a digital representation or computational simulation (i.e. the Digital Twin (DT)) of a physical system is provided and connected to its physical counterpart, for example through the Internet of things as explained in US 2017/286572 A1 for example. Through this connection, the DT typically receives data pertaining to the state of the physical system, such as sensor readings or the like, based on which the digital twin can predict the actual or future status of the physical system, e.g. through simulation.

A DT has been defined as “a set of virtual information constructs that mimics the structure, context and behavior of an individual or unique physical asset that is dynamically updated with data from its physical twin throughout its life-cycle.” Although this definition targets engineering systems, a broad interpretation covers the use of DT in a diverse array of other application areas such as healthcare and information systems. Thus, DTs may be constructed for processes and living entities, and the lifecycle may be the period over which the digital twin is needed to support decision-making. DT may also be used over fixed time periods, such as for surgery or in critical decision points for physical systems. In essence, a digital twin is an in silico model that brings together the technology to map, monitor and control real-world entities by continually receiving and integrating data from a physical twin to provide an up-to-date digital representation of the physical entity.

Such DT technology is also becoming of interest in the medical field, as it provides an approach to more efficient medical care provision. For example, a DT may be built using imaging data of a subject (i.e. patient), e.g. a person suffering from a diagnosed medical condition as captured in the imaging data.

The DT(s) of a subject (i.e. subject-specific computational simulations) may serve a number of purposes. Firstly, the DT(s) (rather than the subject) may be subjected to a number of virtual tests, e.g. treatment plans, to determine which treatment plan is most likely to be successful to the subject. This reduces the number of tests that physically need to be performed on the actual subject (e.g. patient). Secondly, the DT(s) of a subject may be used to predict the onset, treatment (outcome) or development of medical conditions of the subject. That is, the DT(s) of a subject may offer a healthcare professionals advanced visualization and/or physical insights into health information of the subject, thus supporting improved Clinical Decision Support (CDS).

Typically, it is desired that DTs are personalized to better fit and model a subject. However, like any other tool, a DT's utilization and usefulness may depend on the match between a DT functions and a user's skills/capabilities.

For example, considering a digital twin that can generate multiple outputs (e.g. output1=estimation of heart rate variability, output2=oxygen (O₂) supply to an organ), the outputs will be a function of the DT inputs (which can be numerous and varied), and the DT computational model (i.e. code or algorithm) characteristics. However, in practice, different user needs, user capabilities and environmental conditions (i.e., usage context) can arise, resulting in variations in input quality and/or completeness. Also, there are variations due to the context (e.g. subject condition or clinician environment characteristics) which can influence the speed, accuracy or completeness requirements that input or outputs should abide to. Furthermore, different outputs (e.g. output1 and output2) can be influenced differently by the input(s) and model variations, thus resulting in variation in output complexity and/or uncertainty. Such variations in inputs and outputs can negatively impact the reliability and/or accuracy of a DT in practical (e.g. real life) applications.

### SUMMARY OF THE INVENTION

The invention is defined by the claims.

According to examples in accordance with an aspect of the invention, there is provided a control system for controlling a DT of a biological asset of a subject, the system comprising: an interface adapted to obtain: user information describing one or more characteristics of a user of the DT; subject status information describing a medical status of the subject; and historical usage data describing previous usage of the DT; and a control unit configured to generate one or more parameter values for the DT based on the obtained user information, subject status information and historical usage data. It is proposed that aspects of a DT can be controlled (e.g. adapted, modified or otherwise changed) according to its user(s). That is, a DT may be adapted to fit its user(s). Such control of aspects (e.g. elements or components) of a DT may be undertaken automatically or semi-automatically.

Proposed concepts are based on a realization that real-world implementations of clinical decision support systems (CDSS) indicate that it is preferable for DTs to perform reliably and accurately whilst also being flexible enough to adapt to user (e.g. physician) needs and/or usage context (e.g. environment). It may also be preferable that a DT is transparent with regard to how its outputs are calculated and/or what parameters influence the outputs. Embodiments propose to control digital twin components, such as input(s), processing code, or outputs according to a user.

By controlling components or elements of a DT according to a user, embodiments may address the problems associated with: (i) variations in input quality or completeness (resulting from user expertise, knowledge or qualifications, for example); and/or (ii) variations in output complexity or uncertainty (resulting from DT model variations for example). Improved utilization and/or fewer clinical errors may thus be realized by proposed embodiments. Also, by providing a DT user with greater control of speed, accuracy, transparency, and/or interpretability aspects of the digital twin, embodiments may facilitate the provision of more appropriate results.

As a result, systems and methods are proposed for controlling a DT of a biological asset of a subject based on (i) characteristics of a user of the DT; (ii) a status of the subject; and (iii) previous usage of the DT. Such control is facilitated through the generation of one or more parameter values for the DT. For instance, the parameter value(s) may define one or more elements or components of the DT, such as program code, input requirements, output characteristics or a value of a tunable element of the DT. The biological asset may include any biological entity (of different sized and functionalities: cells, tissue, organ, system, any combination of these), or any biological process.

Proposed embodiments may thus provide automatic and dynamic DT recommendation and/or adaption concepts that cater for different users. Further benefits provided by proposed embodiments may include: (i) Enabling a user of the DT to achieve an intended result faster, by facilitating use of right inputs, right code, and right outputs; (ii) Enabling improved accuracy, completeness, and interpretability of the final output; (iii) Reducing errors that may result from incorrect use of the DT and/or a user's lack of ability to interpret DT use; and (iv) Reduction of cost(s) linked to DT usage.

It is also proposed that elements (e.g. components or functions) of a DT may be controlled (e.g. modified or adapted) based on parameter values determined according to embodiments. Further, this may be extended to control operations of connected DTs. Embodiments may therefore support or facilitate DT functionality adaptation, user expertise-based adaptation, DT personalization, uncertainty estimation, input selection, model selection, and/or output selection.

Embodiments may therefore be of particular use in relation to DTs that support clinical decision making. Exemplary usage applications may for example, relate to predicting the onset, treatment (outcome) or development of medical conditions and/or medical procedures. Embodiments may thus be of particular use in relation to medical care management and/or prediction.

By way of example, a proposed embodiment may comprise a control system for a DT having tunable elements (e.g. component or function). The control system may determine one or more parameter values for controlling (e.g. defining or changing) the tunable elements of the DT, and the one more parameter values may be provided via a control signal generated by the control system. For instance, tunable elements of the DT may relate to the DT input(s), DT program code, and/or output characteristics of the DT.

In some embodiments, the control unit may comprise a data analysis component configured to implement at least one of a rule-based algorithm and a machine-learning algorithm for generating one or more parameter values based on the obtained user information, subject status information and historical usage data. For example, the data analysis component may comprise a neural network trained using training inputs and known outputs, wherein the training inputs comprise at least one of user information, subject status information and historical usage data, and wherein the known outputs comprise parameter values for the digital twin. Embodiment may thus employ supervised (or un-supervised) learning concepts which result in improved accuracy and/or confidence.

By way of example, the one or more characteristics of the user comprise at least one of: identification; qualification(s); experience; job title; authorization; physical state; mental state; assigned role and usage preferences. Various characteristics of a user which may influence input quality and/or completeness may thus be leveraged in order to determine how a DT should be controlled (e.g. modified or adapted).

The user may be a provider of input data to the digital twin, such as a person (e.g. nurse or technician) collecting, selecting, controlling or monitoring data for the DT. Such data may be accessed directly, via accessing databases of patient data or sensor signals, or indirectly, where the data is first processed by another algorithm (e.g. patient monitors) and then used by the DT. Alternatively (or additionally), the user may be a consumer of output data provided the digital twin, such as a person that is using the output (e.g. physician or consultant).

In some embodiments, the subject status information comprises at least one of: timing data describing a timing of the delivery of a care provision to the subject; and care data describing a care need of the subject. For instance, the subject status information may comprise information about a measured or perceived stability/status of the subject. Such information may relate to timing aspects of when care is to be provided to the subject (e.g. while a stable ICU patient may need continuous monitoring only, an unstable patient may need immediate treatment). By way of further example, such information may indicate a type of care that is needed, e.g. additional laboratory data, tests, surgery, etc.

Purely by way of example, the historical usage data may comprise at least one of: previous user data describing one or more previous users of the digital twin; previous input data describing one or more previous inputs to the digital twin; previous output data describing one or more previous output of the digital twin; utilization data describing an amount of prior usage of the digital twin; and code data describing previous program code of the digital twin; accuracy data describing an accuracy of previous outputs of the digital twin. In this way, embodiments may leverage information about which users have used the DT twin before and how (e.g. with what inputs, code (i.e. processing models), and outputs, for how long, etc.).

According to some embodiments, the one or more parameter values for the DT comprise: a DT component specification; a program code definition; a confidence value; a decision threshold value; and an access right definition. In this way, embodiments may specify DT usage parameters that determine how a DT output should be generated. That is, embodiments may determine DT specifications relating to input, code, and output requirements. Alternatively, or additionally, embodiments may determine user right specifications relating to functionalities and outputs that may be prioritized or allowed for the DT user.

In an embodiment, the system may be further configured to generate a control instruction (e.g. in the form of a control signal) based on the generated one or more parameter values. In this way, a DT may be controlled, modified or adapted according to results generated by embodiments. Dynamic and/or automated control/adaptation concepts may therefore be realized by proposed embodiments.

Further, proposed concepts may provide a clinical decision support comprising: a digital twin of a biological asset of a subject; and a control system according to a proposed embodiment.

According to another aspect of the invention, there is provided a method for controlling a digital twin of a biological asset of a subject, the method comprising: obtaining: user information describing one or more characteristics of a user of the digital twin; subject status information describing a medical status of the subject; and historical usage data describing previous usage of the digital twin; and generating one or more parameter values for the digital twin based on the obtained user information, subject status information and historical usage data.

Such a method may be computer-implemented. According to examples in accordance with yet another aspect of the invention, there is provided a computer program product comprising computer program code means which, when executed on a computing device having a processing system, cause the processing system to perform all of the steps of the method described above.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 depicts an exemplary embodiment of a system according to an embodiment;

FIG. 2 is a flow diagram of an exemplary implementation according to an embodiment;

FIG. 3 depicts an exemplary implementation for estimating the output generation options for a DT according to an embodiment;

FIG. 4 illustrates an example of how user profiles can be used to determine the allowed boundary conditions according to an embodiment;

FIGS. 5A-5D are screenshots taken from an exemplary clinical platform according to an embodiment;

FIG. 6 is a flow diagram of a method according to an embodiment; and

FIG. 7 is a simplified block diagram of a computer within which one or more parts of an embodiment may be employed.

### DETAILED DESCRIPTION OF THE EMBODIMENTS

The invention will be described with reference to the Figures. It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides concepts for generating one or more parameter values for a DT according to a user. Such concepts may enable adaptation of a DT to a user and may also be extended to automatic and/or dynamic adaptation. That is, embodiments propose that aspects of a DT can be controlled (e.g. adapted, modified or otherwise changed) according to its user(s). Based on such control concepts, a DT may be adapted to fit to its user(s).

In particular, concepts are proposed for controlling a DT of a biological asset of a subject based on (i) characteristics of a user of the DT; (ii) a status of the subject; and (iii) previous usage of the DT. Proposed embodiments may therefore leverage information about a user and a subject to determine how a DT may be adapted. Whereas conventional approaches to improving DTs relate to providing more accurate modelling of a subject, proposed concepts are based on a realization that it may be beneficial to control or adapt a DT according to its user's characteristics, needs and context.

To facilitate description of the proposed concept(s) and embodiments, description provided herein is focused primarily on DTs for healthcare and DTs users with clinical knowledge, e.g. physicians, nurses and other medical professionals. In a single hospital, there are typically clinicians with different expertise (e.g. cardiologists, neurologist, radiologist, etc.), different roles (e.g. nurse, resident, physician, etc.), differing levels of experience (e.g. young doctors), and different specialized training (e.g. trained with robot arms, etc.). Moreover, there may be significant variations across multiple hospitals (e.g. differences in users from one hospital to another). Due to these factors, every professional has a specific context and way of working, and therefore specific needs and requirements. For a potentially powerful tool, such as a DT, to be adopted and used to its full capacity in practice, it is proposed to analyze and adapt DT according to user characteristics. In particular, proposed embodiments described herein present various components of a DT that may be adapted, and how they may be adapted according to the different user characteristics (such as job role and experience level).

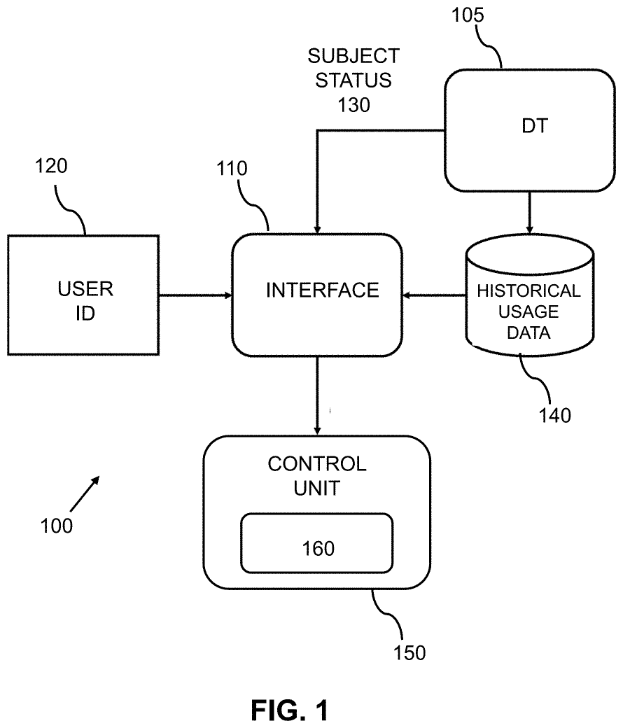

Referring now to FIG. 1, there is depicted an exemplary embodiment of a system according to an embodiment. Specifically, there is depicted a simplified block diagram of a system 100 for controlling a digital twin 105 of a biological asset of a subject. The system comprises an interface 110 and a control unit 150.

The interface 110 is adapted to obtain (e.g. receive or retrieve) user information 120 describing one or more characteristics of a user of the DT 120. By way of example, the one or more characteristics of the user may comprise: identification; qualification(s); experience; job title; usage preferences; physical state; mental state; assigned role and/or authorization. In the example of FIG. 1, the user information comprises use identification data 120, i.e. information about the user(s) going to interact with the DT. In this regard, it is noted that user may be a provider of input data to the DT 105 and/or a consumer of output data provided by the DT.

The interface 110 is also adapted to obtain (e.g. receive/retrieve) subject status information 130 describing a medical status of the subject. By way of example, the subject status information 130 may comprise: timing data describing a timing of the delivery of a care provision to the subject; and/or care data describing a care need of the subject, i.e. information about resource(s) required for the care (e.g. medical staff, equipment, location, etc) and/or the measured, predicted or perceived stability/status of the subject. In the example of FIG. 1, the subject status information 130 is provided to the interface 110 from the DT 105. In other examples, the subject status information 130 may be provided to the interface by a user, a software application (e.g. predictive algorithm), device (e.g. ultrasound), database (e.g. Electronic Medical Records) and/or tool (e.g. subject monitor).

The interface 110 is yet further adapted to obtain historical usage data 140 describing previous usage of the digital twin. Such historical usage data 140 may provide information about which users have used the DT before and how: with what inputs, code (i.e. processing models), and outputs, for how long, etc. For example, the historical usage data 140 is provided from a database 140 and comprises at least one of: previous user data describing one or more previous users of the digital twin; previous input data describing one or more previous inputs to the digital twin; previous output data describing one or more previous output of the digital twin; utilization data describing an amount of prior usage of the digital twin; and/or code data describing previous program code of the digital twin. Historical usage data 140 may be linked to a specific user profile for example.

The interface 110 is configured to provide the obtained information/data to the control unit 150 for processing according to proposed embodiments. The control unit 150 is configured to generate one or more parameter values for the DT 105 based on the obtained user information, subject status information and historical usage data. Specifically, the control unit 150 of FIG. 1 comprises a data analysis component that is configured to implement a machine-learning algorithm for generating one or more parameter values based on the obtained user information, subject status information and historical usage data. The machine-learning algorithm can be implemented as a neural network that is trained using training inputs and known outputs. The training inputs comprise at least one of user information, subject status information and historical usage data, and the known outputs comprise parameter values for the digital twin.

That is, the embodiment of FIG. 1 employs a machine-learning algorithm for generating (e.g. predicting) one or more parameter values for the DT 105 based on the obtained user information, subject status information and historical usage data. The machine-learning algorithm may employ widely-known supervised (or un-supervised) learning concepts/methods and/or conventional neural network architecture that enables accurate prediction of parameter values for the DT 105 based on learnings from prior DT implementations.

Parameter values that may be generated for the DT 105 may include: a digital twin component specification; a program code definition; a confidence value; a decision threshold value; and an access right definition. That is, the control unit 150 may determine values and/or types to be used by one or more elements of the DT 105, and such values may determine or define an implementation of the DT 105, e.g. DT specifications related to input, code, and output requirements. By way of further example, such value may define user right specifications related to functionalities and outputs that would be prioritized or allowed for the DT user. In this way, the DT 105 may be adapted according to the context within which the DT 105 is to be used, e.g. according to both the user and the subject. That is, boundary conditions for running the DT 105, or simulating the subject condition may be determined. Optimization of the DT usage according to user skills and DT capabilities and functionalities may therefore be facilitated.

In some embodiments, the control unit 150 may be further configured to generate a control instruction for a CDSS or a DT based on the generated one or more parameter values. For example, such a control signal may comprise control instructions for controlling elements or components of a DT. In another example, the control signal may comprise control instructions for a display and/or medical equipment, thus enabling the medical equipment to be automatically and/or pre-emptively controlled. In yet further examples, the control unit 150 may be further adapted to derive DT model recommendations. Some embodiments may therefore output information in the form of advice or feedback (e.g. instructions for data collection or instruction to adapt procedures) to a user (e.g. physician).

By way of further description, various implementation examples and/or considerations linked to the aforementioned exemplary embodiment will now be described. (i)(a) Depending on the qualification and experience of the use, input data requirements may differ. For example, if a nurse is experienced with advanced monitoring systems, a direct intra-arterial pressure monitoring (by use of intra-arterial catheter) can be selected for monitoring of a subject's systemic blood pressure. If the nurse is less experienced, a less direct (but also less accurate) measurement with sphygmomanometer with a cuff and Doppler devices can be selected. In the latter case, due to the lower accuracy it may the case that more input data is needed compared to the former case, to reach to the same DT output uncertainty level. (i)(b) One may consider that, for a nurse, the DT outputs generated for hemodynamic monitoring would be: heart rate, systolic and diastolic blood pressures, pulmonary artery pressures, right atrial pressure, cardiac output, and stroke volume, calculated with 95% confidence level. For a physician, additional DT outputs can include: measurement of the “a” and “v” wave of the RA and PAW waveform tracings, mean arterial pressure, cardiac index, stroke volume index, vascular resistance, systemic vascular resistance, pulmonary vascular resistance, and stroke work, calculated with 90% confidence level. (ii)(a) If the subject is experiencing a shock, a care need may be to stabilize the patient as fast as possible, and only then to apply the optimal treatments. In such instances, the role of the DT can be to provide a fast indication of potential shock types (cardiogenic, distributive, or hypovolemic) the patient may be experiencing. (ii)(b) Only when the patient has been stabilized, calculation and display of additional parameters, and suggestions for treatment options, and lab tests can be activated. (iii) Previous (input, code, and output usage) information of the DT can be used for further personalization. For example, a nurse that has been trained to use the DT can be shown all the additional outputs mentioned in (i) above.

To describe an exemplary use of the proposed concept(s), a clinical context will now be considered and different steps in how a DT may be used will described. Specifically, the following will consider first and second instances that the DT is used in, to match a typical workflow where a subject is examined at specific (first and second) instances by a physician or resident, and for the remaining of the time monitored by the nurse.

First use of the DT. At this stage, the output desired by the DT is specified. The desired output can be of descriptive or predictive nature, and it can include a specific parameter, such as hemodynamic stability index (and measurements related to it); (ranked) list of potential diagnoses; or treatment options, etc.

It is assumed that the output can be generated in many different ways. In other words, different inputs and (data processing) models/algorithms can generate the same type of output. That is, the relationship between DT output and other DT components (i.e. DT input and DT code) is not one to one. Moreover, multiple outputs may be generated for the same code and input combination.

Different options for generating the desired output are presented to the user. Notably, however, the presented options are user dependent. In other words, based on categorization of the users, different users may see different options. For instance, the categorization of the users can be based on a single or multiple attributes (such as role, experience level, training received, hospital workflow, tiredness, stress level, preferences, etc.) After the user selects at least one of the options, necessary data collection or calculations can start (enabling the DT output generation).

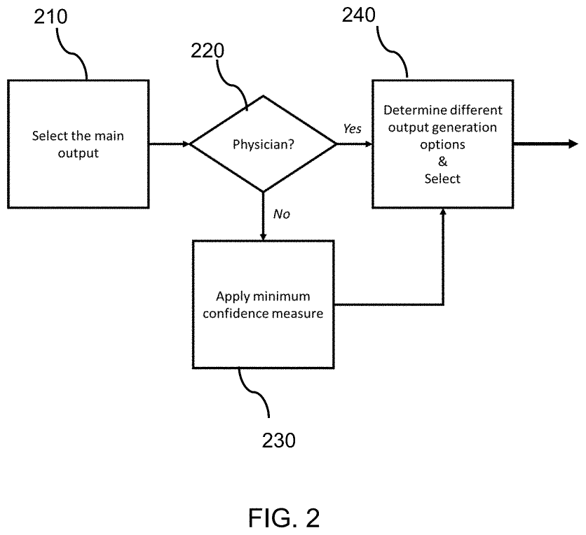

Referring now to FIG. 2, an exemplary implementation flow diagram is shown. In this example implementation method, users are classified in terms of their role, and more specifically, whether users are physicians or not. In step 210, the main output is selected and the method proceeds to step 220 wherein it is determined if the user is a physician or not.

If, in step 220, it is determined that the user is not a physician, the method proceeds to step 230. In step 230, a minimum confidence threshold is employed before proceeding to step 240. If, in step 220, it is determined that the user is a physician, the method proceeds to step 240. In step 240, output generation options are determined and selected. Specifically, in this example, if the user is a physician, the means to generate the output are configured to generate a list with more options compared to a user who is not a physician. This is due to the fact that not being a physician would result in restricted output generation options to only include options that satisfy a defined (stricter) criteria, e.g. depending on the result of employing the minimum confidence threshold level from step 230.

Different options for generating the same output. In a clinical setting, four main output generation features can be considered. These are: (i) Input data and input parameters: In general, any kind of input (machine or human generated, measured or estimated, etc.) that can be used to generate the specified output. In many cases, the input parameters that are accepted by a DT model (i.e. computer program) are well defined and specified. (ii) Estimated output generation time: The time needed to generate the requested output. Note that this can be dependent on many factors, including hardware (e.g. memory, storage, sensors, etc.) or software (e.g. license rights, availability of EMR, etc.) capacity, subject availability/suitability/cooperation (e.g. the subject may be unwilling/unable to be in a position suitable for collection of the required data), or resources availability (for example, in case new laboratory data collection may be needed). Note that the time can be also dependent on the input specifications and/or a capability (e.g. knowledge, familiarity, availability, access, etc.) of the user to operate the DT. (iii) Uncertainty: This relates to the uncertainty level of the output. It can be quantified in terms of different means depending on the processing approach that is employed. For example, confidence intervals, or credible intervals can be used. In addition to the uncertainty level, a measure of the expected bias level can be also included. Note that the uncertainty can be dependent on the input and generation time. (iv) Resources: In some embodiments, the (human, and money) resources required to generate each output can be also indicated. For example, this may be relevant if specific (extra) types of input are needed (such as MRI, CT images, lab tests, for example), or if specific software licenses are required to process and store DT data.

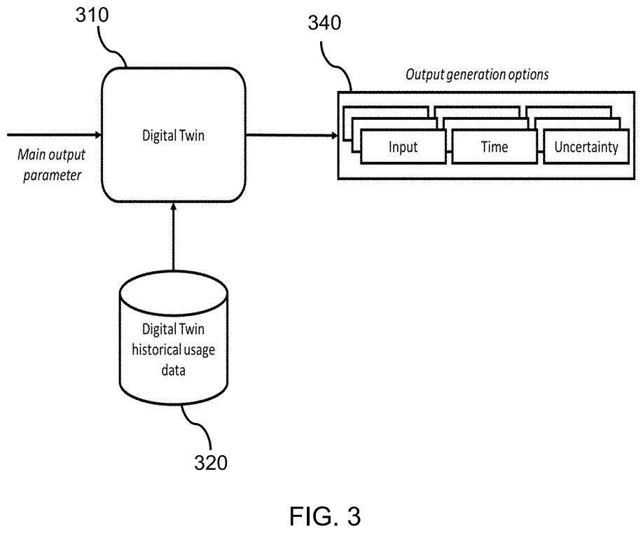

An example implementation for estimating the output generation options for a DT is shown in FIG. 3. As shown in the FIG. 3, one way to determine the output generation options 340 for a DT 310 is based on historical data 320 relating to previous usage of the DT. Such historical usage data 320 may include information about output, input, time, resources and output uncertainty. Using this database of historical usage data 320, a requested output is matched to an output stored in the database, and input candidates to generate the output are determined. The time needed to gather and process the identified inputs is estimated, and accordingly the minimum achievable uncertainty level (taking into account the inputs and time) is estimated.

In case historical usage of the current DT is limited, then historical usage of a similar DTs (similar in terms of input, and algorithm (code), and running environment, and hospital resource(s), and users) and/or a lookup table can be used for estimating the output options 340. For instance, lookup tables may be first provided by the supplier of the DT(s), and, if necessary, can be adapted by a healthcare provider according to guidelines and protocols followed.

Another way to determine the output generation options 340 is by means of computations only. In many cases, the input signal amount, type and quality, and data processing algorithm characteristics can be used to estimate the processing time, and expected uncertainty level, especially when the code is based on bio-physical models.

Example output generation options are shown in Table 1 below.

TABLE 1

| Option | | | confidence |
|---|---|---|---|
| number | input | time | level |
| 1 | Invasive sensor data & lab | 3 min | 99% |
| | analyses: Heart rate, SPO2, | | |
| | arterial blood pressure, | | |
| | body temperature, blood | | |
| | analysis results | | |
| 2 | Non-invasive sensor data & | 4.5 min | 99% |
| | lab results: | | |
| 3 | Non-invasive sensor data & | 3 min | 95% |
| | lab results: | | |
| 4 | Non-invasive sensor data | 2 min | 90% |
| 5 | Non-invasive sensor data | 1 min | 80% |

In this example, the required output is the estimation of the amount of oxygen delivered to the body tissues (in a specific organ). The rate at which oxygen (O2) is supplied to the body tissues is the product of cardiac output (systemic blood flow) and arterial oxygen content. Arterial O2 content is a function of arterial hemoglobin concentration, hemoglobin O2 saturation, and the pressure of O2. Systemic O2 is dependent of many factors, such as O2 transport to lungs, blood, and circulation system. In addition, each organ can respond differently to overcome deficiencies in O2 delivery.

In clinical settings, there are situations where a quick/rapid decision is needed (for example, when a patient is experiencing a hemodynamic shock). In such emergency situations, the time parameter is the main determinant for how the output can be calculated. In case the patient is stabilized, certainty of the output may then become more important (and time less important). It will therefore be appreciated that there may be a continuous trade off between time and uncertainty requirements.

Factors that can influence uncertainty are the input (type, amount, and quality) and the DT model properties (e.g. accuracy). Both of these may be directly linked to time. Reduced order models can lead to fast computations, but may be less accurate, whereas more complex DT models may be very accurate but slow in computation time.

Inputs generated by another algorithm may, for example, be limited by operational constraints of that other algorithm. Certain types of input, such as laboratory data (e.g. blood analyses) can significantly reduce the uncertainty. However, acquiring these takes time. Hospital resources, including the number of people and experience of the caregiver team, may also play a role in the time and uncertainty trade off (mainly because it can significantly influence the process of the input data collection).

It is to be understood that the example of FIG. 3 demonstrates only one particular implementation example, where the main output is used to determine potential input-time-uncertainty options. Variations of this are also possible. For example: (i) In addition to output, a maximum allowed computation time can be also specified, and based on these possible inputs and expected uncertainties can be determined. (ii) In addition to the output, a maximum allowed uncertainty can be specified, and based on these possible inputs and expected times can be determined. (iii) In addition to the output, the inputs can be selected, and based on these the expected time and uncertainty can be determined. (iv) Output, time, and uncertainty can be specified, and input needed for these can be estimated.

User dependent maximum allowed uncertainty. As discussed above, the level of allowed (output) uncertainty may be user-specific. In clinical settings, it can be expected that a physician is more knowledgeable and experienced than a resident or nurse, and may therefore better interpret and utilize higher uncertainties (and complexities) in the DT output (when compared to a resident or nurse). Similarly, a resident that has received DT usage training, or used the DT before, can be expected to cope with higher uncertainties compared to a resident with little (or zero) DT usage training.

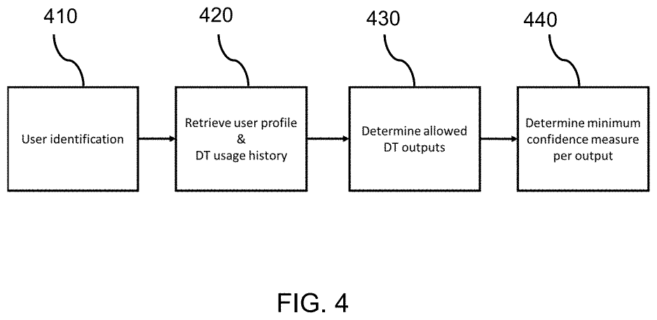

An example of how user profiles can be used to determine the allowed boundary conditions (in terms of output uncertainty, and also potentially linked to other parameters such as input data types, and computational resources, (reduced) bio-physical models selection) is shown in FIG. 4. In particular, FIG. 4 depicts an example where user profile and DT usage history (i.e. user information and historical usage data) is used to determine how the user can use the DT.

In step 410, user identification is obtained and the method proceeds to step 420. In step 420, the user identification from step 410 is used to retrieve (i) a user profile for the identified user and (ii) historical usage data for the identified user. Based on the retrieved user profile and historical usage data, a set of permitted/allowed DT outputs is determined in step 430 (e.g. using rule-based algorithms). Finally, in step 440, a minimum confidence measure is determined for each of the allowed DT outputs.

For example, an emergency department physician (or a clinician empowered with rights by the physician) may be allowed to generate many outputs, with various degrees of uncertainties to help in deciding better where (to which hospital unit) to submit the admission request. The admitting physicians can be allowed to generate only the outputs required for their units, to access the suitability of admission. As another example: A nurse can be shown the basic outputs only when they become statistically significant at 95% confidence level, while for a resident a default interface can also include additional outputs, and their values will be displayed together with their corresponding confidence intervals calculated at 90% confidence level.

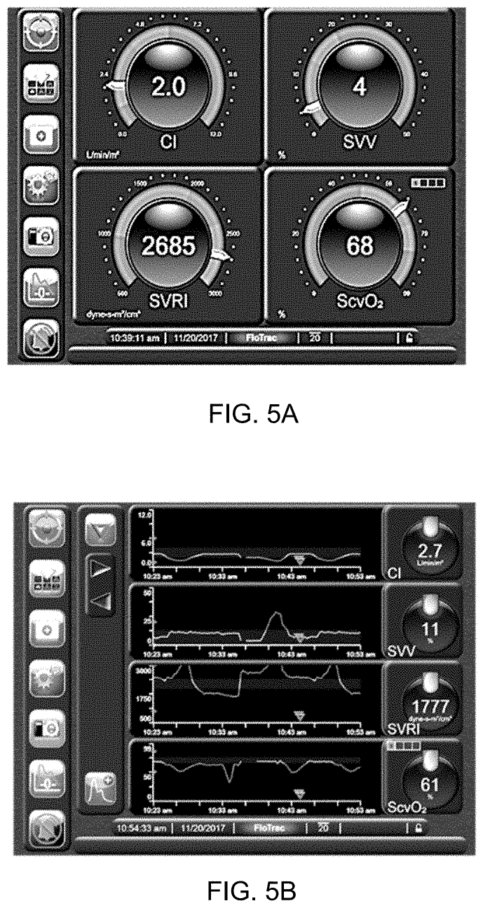

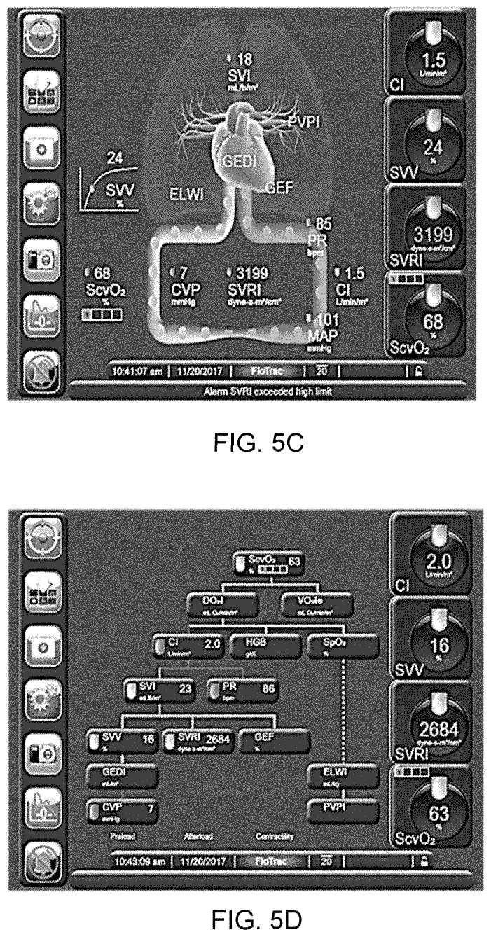

Second use of DT. During the first examination, the examining clinician may have selected the main outputs that the clinical support system (such as a DT) needs to generate. In most cases, the main output can be accompanied with other additional parameters. For example, in addition to the oxygen delivery amount estimation, the output may include organ specific oxygen demand and delivery indicators, estimation of hemodynamic shock risks including a patient stability indicator, suggestion for possible measurements, treatments, medications, and so on. These outputs which differ from the main output are referred to as additional outputs. Examples of various parameters that can be measured, estimated and visualized for hemodynamic monitoring are shown in FIGS. 5A-5D. Specifically, FIGS. 5A-5D are screenshots taken from an exemplary clinical platform.

According to the proposed concepts, depending on the user characteristics, the displayed information characteristics (type, detail, amount, frequency) vary. However, it is preferable to do this in a manner that does not negatively interfere with the best possible treatment the subject (e.g. patient) can or should receive. More specifically, embodiments may be configured so that users with potentially less rights or knowledge and experience (e.g. nurses or medical students) may can still access all relevant information to treat the patient. To achieve this, some embodiments propose that physicians (e.g. users with a first user skillset or qualification) are able to access and modify the main output as well as all other additional outputs at all times. Medical students (e.g. users with a second user skillset of no qualification) are also able to access the main outputs at all times (regardless of whether their uncertainty suits to the medical students' profile). However, furthermore, only the additional outputs satisfying the student specific uncertainty threshold will be shown. If the main output meets the uncertainty requirements fitting to the student's profile, there may be provided an option specify a new output.

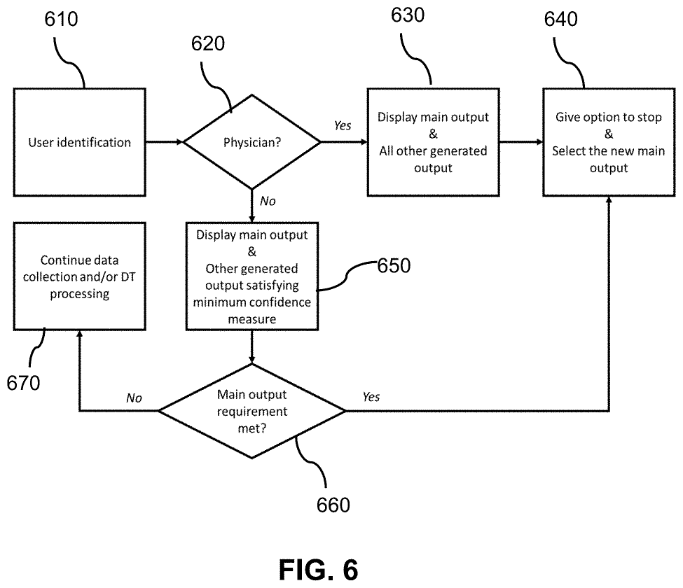

An example flow diagram according to an embodiment is shown in FIG. 6. As illustrated in FIG. 6, if the user has qualifications of a physician, then both main and additional output are shown, otherwise only the main output, and additional output satisfying a user profile specific criteria are displayed. In more detail, the method begins in step 610 with identification of the user. Next, in step 620, it is determined if the identified user has qualifications or rights of a physician. If it is determined in step 620 that the identified user has qualifications of a physician, the method proceeds to step 630, wherein then both main and additional output(s) are displayed to the identified user. Then in step 640, the user is provided with an option select a new main output (e.g. via a user interface).

Conversely, if it is determined in step 620 that the identified user does not have qualifications of a physician, the method proceeds to step 650, wherein only the main output and additional output(s) satisfying a user profile specific criteria are displayed to the user. Then in step 660 it is determined whether the main output requirement is met. If it is, the method proceeds to step 640. If it is not, the method proceeds to step 670 wherein data collection and/or DT processing it continued. Yet further exemplary embodiments will now be described in the following title sections.

Selecting the DT version to run based on the subject status, workflow and guidelines information. Subject status may be classified as emergency or non-emergency. In emergency situations, it may be preferable to provide a DT output as soon as possible, with a reasonable uncertainty or accuracy. Conversely, in non-emergency situations, it may be preferable to generate the most accurate or certain output, within a reasonable time. Workflow information may include knowledge of the number and expertise of clinicians, as well availability of the devices, rooms, bed, and other resources specific to the environment where subject care will be provided, and information of the computational environment where DT will run. Guidelines information includes the protocol and steps that should be followed. DT output generation speed, output accuracy or uncertainty, and required resources (e.g. time, people, utilities, and hardware) to complete processing (e.g. to collect data, to process the data, to interpret the results, etc.) can significantly vary depending on the input, code, and output features of the digital twin.

In this embodiment, it is proposed to employ a database (e.g. a lookup table) with all such information stored. By referencing the database, the appropriate version of the DT that matches to a user, subject and workflow parameters can be selected and used. Providing for such a selection of DT configuration (e.g. from a database or lookup table of DT parameter values) may assist in the implementation embodiments described above. For example, if it is known in advance that the facilities to collect new data (e.g. MRI, x-ray) may not be immediately available, code of DT that can cope with lack of such data may be selected from the outset. Similarly, if it is known that caregiver nurse is inexperienced (e.g. few months of experience), the DT can be configured to compute only basic vitals (e.g. oxygen demand), instead of advanced metrics (hemodynamic instability index, predictive measures, etc.).

Selecting users based on current DT status. In some situations, such as emergency conditions, it may not be possible to collect/use more input data or more complex models to improve the uncertainty of the DT output. In these situations, in order to prevent medical error (e.g. due to over-reliance on the DT output), or to make sure that the DT outputs are utilized (since they may not be used at all if a user does not understand them or is not comfortable with them) it may be desirable to determine the appropriate user(s) that can properly understand and use the DT output(s). This may be achieved by employing a database of DT models and outputs that is further annotated or classified with meta-data linking them to the expertise, and later alerting and involving the experts based on the DT characteristics and current status.

Linking the DT output features to alert fatigue or tiredness. The amount of information displayed from a DT may be linked to alarm fatigue. If the number and frequency of the alarms generated by the DT or other devices is tracked, it may be assumed that with time the users may become fatigued and therefore start interpreting and using the DT output differently. Therefore, it may be desirable to adapt the level and amount of DT output details accordingly. One way to do so, is to display only the main output, and only display any other additional outputs when they are relevant and with very high accuracy. Another approach is to display more details and more outputs at the beginning of the shift of the nurse, and gradually decrease the amount and details of the information, by only displaying the relevant outputs for the current moment (for example, after a particular drug administration it may be more important to monitor the blood pressure, and less important to monitor the body temperature, and in this case the DT can display both blood pressure and body temperature related analyses or prediction at the start of the shift, and later only display the blood pressure predictions).

From the above-described embodiments and examples, it will be understood that the proposed concepts are particular relevant to medical facilities (e.g. hospitals) where many users may be involved in a subject care process. In particular, embodiments may be of yet further relevance to academic hospitals. It is to be understood that the above examples and embodiments are only illustrative and the ordering of one or more of the steps may be modified in alternative embodiments.

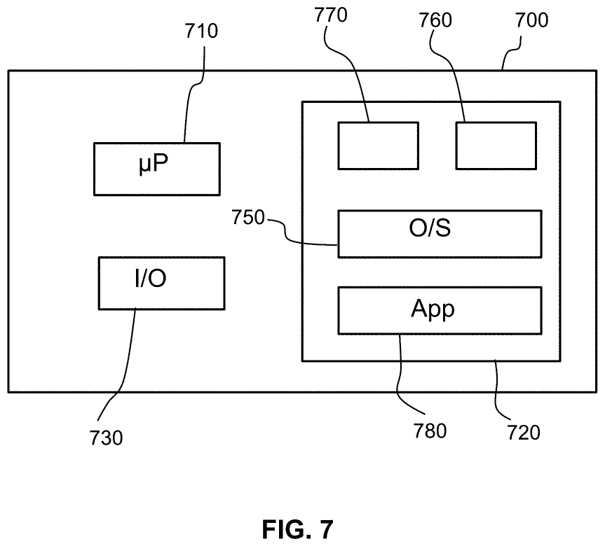

By way of further example, FIG. 7 illustrates an example of a computer 700 within which one or more parts of an embodiment may be employed. Various operations discussed above may utilize the capabilities of the computer 700. For example, one or more parts of a system for controlling a DT of a biological asset of a subject may be incorporated in any element, module, application, and/or component discussed herein. In this regard, it is to be understood that system functional blocks can run on a single computer or may be distributed over several computers and locations connected across a cloud-based system (e.g. connected via internet). That is, at least part of the DTs and data may be stored and executed in one or more cloud-based systems, of which the computer 700 may be part.

The computer 700 includes, but is not limited to, PCs, workstations, laptops, PDAs, palm devices, servers, storages, and the like. Generally, in terms of hardware architecture, the computer 700 may include one or more processors 710, memory 720, and one or more I/O devices 730 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 710 is a hardware device for executing software that can be stored in the memory 720. The processor 710 can be virtually any custom made or commercially available processor, a central processing unit (CPU), a digital signal processor (DSP), or an auxiliary processor among several processors associated with the computer 700, and the processor 710 may be a semiconductor based microprocessor (in the form of a microchip) or a microprocessor.

The memory 720 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 720 may incorporate electronic, magnetic, optical, and/or other types of storage media. Note that the memory 720 can have a distributed architecture, where various components are situated remote from one another, but can be accessed by the processor 710.

The software in the memory 720 may include one or more separate programs, each of which comprises an ordered listing of executable instructions for implementing logical functions. The software in the memory 720 includes a suitable operating system (O/S) 750, compiler 760, source code 770, and one or more applications 780 in accordance with exemplary embodiments. As illustrated, the application 780 comprises numerous functional components for implementing the features and operations of the exemplary embodiments. The application 780 of the computer 700 may represent various applications, computational units, logic, functional units, processes, operations, virtual entities, and/or modules in accordance with exemplary embodiments, but the application 780 is not meant to be a limitation.

The operating system 750 controls the execution of other computer programs, and provides scheduling, input-output control, file and data management, memory management, and communication control and related services. It is contemplated by the inventors that the application 780 for implementing exemplary embodiments may be applicable on all commercially available operating systems.

Application 780 may be a source program, executable program (object code), script, or any other entity comprising a set of instructions to be performed. When a source program, then the program is usually translated via a compiler (such as the compiler 760), assembler, interpreter, or the like, which may or may not be included within the memory 720, so as to operate properly in connection with the O/S 750. Furthermore, the application 780 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 730 may include input devices such as, for example but not limited to, a mouse, keyboard, scanner, microphone, camera, etc. Furthermore, the I/O devices 730 may also include output devices, for example but not limited to a printer, display, etc. Finally, the I/O devices 730 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 730 also include components for communicating over various networks, such as the Internet or intranet.

If the computer 700 is a PC, workstation, intelligent device or the like, the software in the memory 720 may further include a basic input output system (BIOS) (omitted for simplicity). The BIOS is a set of essential software routines that initialize and test hardware at startup, start the O/S 750, and support the transfer of data among the hardware devices. The BIOS is stored in some type of read-only-memory, such as ROM, PROM, EPROM, EEPROM or the like, so that the BIOS can be executed when the computer 700 is activated.

When the computer 700 is in operation, the processor 710 is configured to execute software stored within the memory 720, to communicate data to and from the memory 720, and to generally control operations of the computer 370 pursuant to the software. The application 780 and the O/S 750 are read, in whole or in part, by the processor 710, perhaps buffered within the processor 710, and then executed.

When the application 780 is implemented in software it should be noted that the application 780 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method. The application 780 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

The present invention may be a system, a method, and/or a computer program product. The computer program product may include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. A single processor or other unit may fulfill the functions of several items recited in the claims.

It will be understood that the disclosed methods are computer-implemented methods. As such, there is also proposed a concept of a computer program comprising code means for implementing any described method when said program is run on a processing system. The skilled person would be readily capable of developing a processor for carrying out any herein described method. Thus, each step of a flow chart may represent a different action performed by a processor, and may be performed by a respective module of the processing processor.

As discussed above, the system makes use of a processor to perform the data processing. The processor can be implemented in numerous ways, with software and/or hardware, to perform the various functions required. The processor typically employs one or more microprocessors that may be programmed using software (e.g. microcode) to perform the required functions. The processor may be implemented as a combination of dedicated hardware to perform some functions and one or more programmed microprocessors and associated circuitry to perform other functions. Examples of circuitry that may be employed in various embodiments of the present disclosure include, but are not limited to, conventional microprocessors, application specific integrated circuits (ASICs), and field-programmable gate arrays (FPGAs).

In various implementations, the processor may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims.

The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted that the term “adapted to” is intended to be equivalent to the term “configured to”. Any reference signs in the claims should not be construed as limiting the scope.

1. A control system for controlling a digital twin of a biological asset of a subject, the system comprising:
an interface adapted to obtain:
user information describing one or more characteristics of a user of the digital twin;
subject status information describing a medical status of the subject; and
historical usage data describing previous usage of the digital twin; and
a control unit configured to generate one or more parameter values for the digital twin based on the obtained user information, subject status information and historical usage data.

2. The system of claim 1, wherein the control unit comprises a data analysis component configured to implement at least one of a rule-based algorithm and a machine-learning algorithm for generating one or more parameter values based on the obtained user information, subject status information and historical usage data.

3. The system of claim 1, wherein the data analysis component comprises a neural network trained using training inputs and known outputs, wherein the training inputs comprise at least one of user information, subject status information and historical usage data, and wherein the known outputs comprise parameter values for the digital twin.

4. The system of claim 1, wherein the one or more characteristics of the user comprise at least one of:
identification;
qualification(s);
experience;
job title;
authorization;
usage preferences;
physical state;
mental state; and
assigned role.

5. The system of claim 1, wherein the user is at least one of: a provider of input data to the digital twin; and a consumer of output data provided by the digital twin.

6. The system of claim 1, wherein the subject status information comprises at least one of:
timing data describing a timing of the delivery of a care provision to the subject; and
care data describing a care need of the subject.

7. The system of claim 1, wherein the historical usage data comprises at least one of:
previous user data describing one or more previous users of the digital twin;
previous input data describing one or more previous inputs to the digital twin;
previous output data describing one or more previous output of the digital twin;
utilization data describing an amount of prior usage of the digital twin;
model data describing one or more models of the digital twin;
clinical data describing previous medical conditions of the subject linked to the digital twin;
clinical guideline data describing previous clinical guidelines and protocols linked to previous inputs and/or outputs of the digital twin; and
code data describing previous program code of the digital twin.

8. The system of claim 1, wherein the one or more parameter values for the digital twin comprise:
a digital twin component specification;
a program code definition;
a confidence value;
a decision threshold value; and
an access right definition.

9. A clinical decision support comprising:
a digital twin of a biological asset of a subject; and
a control system for adapting the digital twin according to claim 1.

10. A method for controlling a digital twin of a biological asset of a subject, the method comprising:
obtaining:
user information describing one or more characteristics of a user of the digital twin;
subject status information describing a medical status of the subject; and
historical usage data describing previous usage of the digital twin; and
generating one or more parameter values for the digital twin based on the obtained user information, subject status information and historical usage data.

11. The method of claim 10, wherein generating one or more parameter values comprises:
executing at least one of a rule-based algorithm and a machine-learning algorithm for generating one or more parameter values based on the obtained user information, subject status information and historical usage data.

12. The method of claim 10, wherein the user is at least one of: a provider of input data to the digital twin; and a consumer of output data provided by the digital twin.

13. The method of claim 10, wherein the one or more characteristics of the user comprise at least one of:
identification:
qualification(s);
experience;
job title;
authorization;
user preferences;
physical state;
mental state; and
assigned role.

14. The method of claim 10, wherein the one or more parameter values for the digital twin comprise:
a digital twin component specification;
a program code definition;
a confidence value;
a decision threshold value; and
an access right definition.

15. A computer program product comprising computer program code means which, when executed on a computing device having a processing system, cause the processing system to perform all of the steps of the method according to claim 10.
